(19) United States
(12) Patent Application Publication
(10) Pub. No.: US 2002/0176882 A1
(43) Pub. Date: Nov. 28, 2002

(54) Additive the improvement and/or stabilization of the keeping quality of microbially perishable products

(76) Inventor: Jorg Peter Schur, Wegberg (DE)

Correspondence Address: DANN DORFMAN HERRELL & SKILLMAN, SUITE 720, 1601 MARKET STREET, PHILADELPHIA, PA, 19103-2307, US

(21) Appl. No.: 10/103,396

(22) Filed: Mar. 20, 2002

Related U.S. Application Data
(63) Continuation of application No. 09/446,479, filed on Mar. 10, 2000.
Application No. 09/446,479 is a 371 of international application No. PCT/EP98/03788, filed on Jun. 22, 1998.

(30) Foreign Application Priority Data
Jun. 23, 1997 (DE) 19726429A1

(51) Int. Cl. A61K 31/05; A61K 31/045; A61K 47/00

(52) U.S. Cl. 424/439; 514/729; 514/731

(57) ABSTRACT

The invention relates to a method for improving the storage life of and/or stabilizing microbially perishable products by adding additives, and to a corresponding additive

The present invention relates to a method and an additive and its use for the improvement and/or stabilization of the keeping quality of microbially perishable products by the addition of additives.

Industrially processed food and feed products, cosmetics,, pharmaceuticals and other products susceptible to microbial perishing must be storable for not too short a period of time in order to reach the consumer in an unperished state after shipping and distribution on the usual pathways. In addition, the consumer even expects the acquired product not to perish immediately after having been bought, but to be storable for some days or weeks, depending on the kind of product.

If not treated, most food and feed products would perish within a few days since fungi and/or bacteria could thrive without any hindrance, at most impeded by cooling, on an ideal, for them, substrate. Typical examples include the perishing of bread from molds, e.g., Aspergillus niger, of meat products (e.g., sausages) from enterobacteria or lactobacilli, the contamination of poultry by Salmonellae, and many more. Since fungi including yeasts or their spores, Gram-positive and Gram-negative bacteria are present in every place except where a sterile environment has been created by particular expensive measures, which cannot be applied industrially for economic reasons, appropriate countermeasures must be taken.

Therefore, conventionally, food and feed products, cosmetics, pharmaceuticals, paints, papers and pulps and other perishable products are rendered storable using preservatives, which appear in the Codex Alimentarius list of the Food and Agriculture Organisation (FAO/WHO Food Standard Programme) in Division 3, Food Additives, 3.73, Preservatives, as “synthetic preservatives” and are mostly employed in the form of chemical monosubstances or their combinations.

From the prior art, a wide variety of additives for preserving perishable products has been known. These include, for example, additives based on flavoring agents, alcohols, organic acids, aldehydes, phenolics and essential oils. Such compositions are described, for example, in the U.S. Pat. Nos. 4,446,161, 4,927,651, WO 94/14,414, GB 172993 and DE-OS 3138277, and in E. Lück (Chemische Lebensmittelkonservierung, page 1977, 1986, Springer-Verlag).

The preservatives appearing in the mentioned list are bacteriostatically and/or fungistatically active and substantially improve the keeping quality. However, they are refused by many consumers since their effects on the consumer's health are not known, and adverse effects, especially for a repeated uptake over an extended period of time, cannot be excluded. Another disadvantage is that all methods known to date are based on pH or a_{w} value changes.

One particular disadvantage of these preservatives is their being added to the food product in high concentrations, as a rule. Thus, relatively large amounts of these substances also arrive in the human body when the food is ingested. This results in responses in the form of allergic diseases, which today are often met at a high incidence.

An alternative to the preservation by the addition of synthetic preservatives is the thermal inactivation of germs, e.g., by pasteurizing. Pasteurizing is a thermal treatment at 70 to 85° C. with an exposure time of from 30 to 120 minutes.

Pasteurization substantially improves the keeping quality of thus treated products, but is technically complicated and consumes very much energy. In addition, the viability of spores is often not or only very incompletely eliminated. Also, for thermally sensitive products, pasteurization cannot be used, or it leads to a significant loss in quality since the “degree of freshness” of the pasteurized product will be reduced, at least by the second thermal treatment (at up to 85° C.), which is often necessary. In addition, it is often just the valuable components of food products, cosmetics or pharmaceuticals, e.g., vitamins, amino acids and many pharmaceutically active ingredients, which are thermolabile, so that a thermal treatment under the usual pasteurization conditions is precluded.

Another possibility for improving the keeping quality is to place the product susceptible to perishing in an air-tight package under nitrogen or CO₂, or to supply it in vacuum packages, as is done, for example, with ground coffee. However, these processes are expensive and tedious and thus, for many food products, cannot be used.

Accordingly, it has been the object of the present invention to provide an additive for the improvement of the keeping quality and/or stabilization of microbially perishable products by the addition of additives, which additive lacks the mentioned drawbacks of the prior art.

This object is achieved by the additive's being a mixture containing

a) polyphenol and

b) at least one GRAS flavor alcohol; or

c) benzyl alcohol and

d) at least one other GRAS flavor alcohol;

the mixing ratio of components a) to b) or c) to d) being from 1:1 to 1:10,000 or from 10,000:1 to 1:1, preferably from 1:1 to 1:1000 or from 1000:1 to 1:1. More preferably, the mixing ratio is from 1:1 to 1:100 or from 100:1 to 1:1.

In the following, the substances which may be preferably used according to the invention are further described in more detail:

As the polyphenol (components a)), there may also be used their possible derivatives, salts, acids, esters, oxidases, free and etherified, natural. Examples of useful compounds include pyrocatechol, resorcinol, hydroquinone, phloroglucinol, pyrogallol, hexahydroxybenzene, usnic acid, acylpolyphenols, lignins, anthocyanidines, flavones, catechols, tannins, gallic acid derivatives, carnosol, carnosolic acid, 2,5-dihydroxyphenylcarboxylic and 2,5-dihydroxyphenylalkanecarboxylic, substitutions, derivatives, salts, esters, amides, caffeic acid, esters, amides, gallotannin, tannic acids, pyrogallol, gallotannic acids, flavonoids: flavone, flavonol, isoflavone, gossypetin, myricetin, robinetin, apigenin, morin, taxifolin, eriodictyol, naringin, rutin, hesperidin, troxerutin, chrysin, tangeritin, luteolin, epigallocatechol gallate, quercetin, fisetin, kaempferol, galangin, rotenoids, aurones, flavonols, flavonediols, extracts, e.g., from Camellia, Primula.

According to the invention, the mentioned polyphenols are used in combination with GRAS flavor alcohols. GRAS flavor alcohols have been recognized by the FDA as commercially safe for use in foods (G.R.A.S.=generally recognized as safe in food). This distinguishes the alcohols according to the invention, especially from those employed in U.S. Pat. No. 4,446,161.

The mentioned GRAS flavor alcohols are those alcohols which are mentioned in the FEMA/FDA GRAS Flavour Substances Lists GRAS 3-15 No. 2001-3815 (as of 1997). This list contains natural and synthetic flavoring agents approved by the American public health authority, FDA, for use in foods (FDA Regulation 21 CFR 172.515 (Synthetic Flavoring Substances and Adjuvants) and FDA Regulation 21 CFR 182.20 (Natural Flavoring Substances and Adjuvants)).

According to the invention, component a) may be used with one or more GRAS flavor alcohols (components b)). According to the invention, it is preferred to use one or two GRAS flavor alcohols. It is particularly preferred to use two GRAS flavor alcohols.

In detail, the following GRAS flavor alcohols may be employed, for example (components a), c), d)): benzyl alcohol, acetoin (acetylmethylcarbinol), ethyl alcohol (ethanol), propyl alcohol (1-propanol), iso-propyl alcohol (2-propanol, isopropanol), propylene glycol, glycerol, n-butyl alcohol (n-propyl carbinol), iso-butyl alcohol (2-methyl-1-propanol), hexyl alcohol (hexanol), L-menthol, octyl alcohol (n-octanol), cinnamyl alcohol (3-phenyl-2-propene-1-ol), α-methylbenzyl alcohol (1-phenylethanol), heptyl alcohol (heptanol), n-amyl alcohol (1-pentanol), iso-amyl alcohol (3-methyl-1-butanol), anisic alcohol (4-methoxybenzyl alcohol, p-anisic alcohol), citronellol, n-decyl alcohol (n-decanol), geraniol, β-γ-hexenol (3-hexenol), lauryl alcohol (dodecanol), linalool, nerolidol, nonadieneol (2,6-nonadiene-1-ol), nonyl alcohol (nonanol-1), rhodinol, terpineol, borneol, clineol (eucalyptol), anisole, cuminyl alcohol (cuminol), 10-undecene-1-ol, 1-hexadecanol.

The GRAS flavor alcohols which are particularly preferred according to the invention include benzyl alcohol. Thus, one particularly preferred combination contains polyphenol (component a)) and benzyl alcohol (component b)). This mixture may optionally contain further GRAS flavor alcohols.

According to the invention, it is also possible to use GRAS flavor alcohols (component d)) instead of the polyphenol, i.e., benzyl alcohol (component c)) may be used in admixture with other GRAS flavor alcohols (component d)).

The mixing ratio of polyphenol (component a)) or benzyl alcohol (component c)) to the GRAS flavor alcohols (components b) and d)) is preferably from 1:1 to 1:10,000 or from 10,000 :1 to 1:1, more preferably from 1:1 to 1:1000 or from 1000:1 to 1:1, even more preferably from 1:1 to 1:100 or from 100:1 to 1:1.

However, various alcohols can be additionally employed in components a), b), c) and d). These are preferably monohydric or polyhydric alcohols containing from 2 to 10 carbon atoms, preferably from 2 to 7 carbon atoms.

Preferably, polyphenol and alcohols are used in such amounts that the mixing ratio of alcohol to polyphenol is from 1:1 to 1:1000, or that of polyphenol to alcohols is from 1:1 to 1:1000, especially from 1:1 to 1:100 or from 100:1 to 1:1.

The components of the additive according to the invention mentioned in the following are flavoring agents recognized in the FEMA/FDA GRAS Flavour Substances List as GRAS (generally recognized as safe in food) 3-15 No. 2001-3815 (as of 1997).

Further, in the additive according to the invention, acids and/or their physiologically acceptable salts can be employed. Preferably, organic acids and/or their salts are used. These are preferably compounds containing from 1 to 15 carbon atoms, preferably from 2 to 10 carbon atoms.

In detail, the following acids may be used, for example (component e)):

Acetic acid, aconitic acid, adipic acid, formic acid, malic acid (1-hydroxysuccinic acid), capronic acid, hydrocinnamic acid (3-phenyl-1-propionic acid), pelargonic acid (nonanoic acid), lactic acid (2-hydroxypropionic acid), phenoxyacetic acid (glycolic acid phenyl ether), phenylacetic acid (α-toluenic acid), valeric acid (pentanoic acid), iso-valeric acid (3-methylbutyric acid), cinnamic acid (3-phenylpropenoic acid), citric acid, mandelic acid (hydroxyphenylacetic acid), tartaric acid (2,3-dihydroxybutanedioic acid; 2,3-dihydroxysuccinic acid), fumaric acid. Lactic acid, for example, is preferred.

Further, the following compounds may be used as component f) in the additive according to the invention:

As phenols, there may be used, for example, thymol, methyleugenol, acetyleugenol, safrol, eugenol, isoeugenol, anethole, phenol, methylchavicol (estragol; 3-4-methoxyphenyl-1-propene), carvacrol, α-bisabolol, fornesol, anisole (methoxybenzene), propenylguaethol (5-propenyl-2-ethoxyphenol). As acetates (component g)), there may be used, for example, iso-amyl acetate (3-methyl-1-butyl acetate), benzyl acetate, benzylphenyl acetate, n-butyl acetate, cinnamyl acetate (3-phenylpropenyl acetate), citronellyl acetate, ethyl acetate (acetic ester), eugenol acetate (acetyleugenol), geranyl acetate, hexyl acetate (hexanyl ethanoate), hydrocinnamyl acetate (3-phenylpropyl acetate), linalyl acetate, octyl acetate, phenylethyl acetate, terpinyl acetate, triacetin (glyceryl triacetate), potassium acetate, sodium acetate, sodium diacetate, calcium acetate.

As an ester (component h)), there may be used allicin, for example.

As terpenes (component i)), there may be used, for example, camphor, limonene, β-caryophyllene.

The acetals (component j)) which can be used include, e.g., acetal, acetaldehyde dibutyl acetal, acetaldehyde dipropyl acetal, acetaldehyde phenethyl propyl acetal, cinnamic aldehyde ethylene glycol acetal, decanal dimethyl acetal, heptanal dimethyl acetal, heptanal glyceryl acetal, benzaldehyde propylene glycol acetal.

Aldehydes (component k)) may also be used, e.g., acetaldehyde, anisic aldehyde, benzaldehyde, iso-butyl aldehyde (methyl-1-propanal), citral, citronellal, n-capraldehyde (n-decanal), ethylvanillin, furfurol, heliotropin (piperonal), heptyl aldehyde (heptanal), hexyl aldehyde (hexanal), 2-hexenal (β-propylacrolein), hydrocinnamic aldehyde (3-phenyl-1-propanal), lauryl aldehyde (dodecanal), nonyl aldehyde (n-nonanal), octyl aldehyde (n-octanal), phenylacetaldehyde (1-oxo-2-phenylethane), propionaldehyde (propanal), vanillin, cinnamic aldehyde (3-phenylpropenal), perillaldehyde, cuminaldehyde.

Preferably, according to the invention, solubilizers (component 1)) are also present as a component in the additive. Namely, the additives used according to the invention are flavoring agents, in principle. Most of the flavoring agents included in the GRAS FEMA list are not water-soluble, i.e., they are hydrophobic. If employed in food products which predominantly contain lipids, they can be directly used without a solvent due to their lipophilic character. However, the proportion of lipophilic foods is relatively low. In order to be able to display their activity in the mostly hydrophilic food or feed products, cosmetics or pharmaceuticals, they are preferably used in connection with a water-soluble solubilizer, preferably glycerol, propylene glycol, water, edible oils or fats.

According to the invention, there may also be used, for example, the following essential oils and/or alcoholic or glycolic extracts or extracts obtained by CO₂ high pressure methods (component m)) from the plants:

a) oils or extracts having a high content of alcohols: melissa, coriander, cardamon, eucalyptus;

b) oils or extracts having a high content of aldehydes: Eucalyptus citriodora, cinnamon, lemon, lemon grass, melissa, citronella, lime, orange;

c) oils or extracts having a high content of phenols: origanum, thyme, rosemary, orange, clove, fennel, camphor, mandarin, anise, cascarilla, estragon and pimento;

d) oils or extracts having a high content of acetates: lavender;

e) oils or extracts having a high content of esters: mustard, onion, garlic;

f) oils or extracts having a high content of terpenes: pepper, bitter orange, caraway, dill, lemon, peppermint, nutmeg apple.

The mixing ratio of components a) or c) to b), d), e), f), g), h), j), k), l), m) may respectively be from 1:1 to 1:10,000 or from 10,000:1 to 1:1, preferably from 1:1 to 1:1000 or from 1000:1 to 1:1.

The additives described are preferably used for the improvement and stabilization of the keeping quality of the following groups of food products:

Breads, pastries, improvers, baking powders, blancmange powders, beverages, dietetic food products, essences, delicatessen, fish and fish products, potatoes and products based on potatoes, spices, flours and meals, margarine, fruits and vegetables and products based on fruits and vegetables, pickled preserves, starch products, sweets, soups, pasta food, meat and meat products, milk, dairy and cheese products, poultry and poultry products, oils, fats and products containing oils or fats.

The additives according to the invention are effective, in particular, against molds, yeasts and bacteria (Gram-positive and Gram-negative). They are excellently effective, in particular, against pathogens (Enterobacteriaceae, e.g., E. coli, Salmonella, Enterococci, e.g., Staphylococci, Streptococci) and also against perishing-causing agents, such as lactic-acid bacteria, e.g., Lactobacillus vulgaris, molds, e.g., Aspergillus niger, yeasts, e.g., Endomyces tibuliger. Also, the additives according to the invention have a reductive effect against viruses and microbial toxins, such as aflatoxins, enterotoxins.

The additives are preferably added to the microbially perishable product in amounts of from 1 ppm to 10% by weight, preferably from 1 ppm to 1.0% by weight. Particularly preferred amounts are from 0.001% by weight to 0.5% by weight. Especially preferred amounts are from 0.002% by weight to 0.25% by weight.

According to the invention, it is surprising that the effect of the additives according to the invention can be seen when concentrations as low as those mentioned are used. This is all the more surprising since the food products treated with the additives according to the invention have a significantly higher keeping quality than the perishable products treated with conventional preservatives.

It is also surprising that the advantages described are seen even for microbial exposure times of less than 24 h, especially less than 60 minutes, preferably from 1 to 60 minutes, most preferably from 5 to 15 minutes.

Surprisingly, the additives according to the invention do not result in any disadvantages in the taste, smell or color of the treated food product. One particular advantage over the prior art is that no shifts of the pH or a_{w} value is observed, i.e., the activity of the additives employed is surprisingly independent of the pH and a_{w} values. It is also surprising that the additives can be used irrespective of humidity, fat, protein and carbohydrate contents. Finally, the combinations according to the invention are insensitive to temperature variations within a range of from −30° C. to 200° C., i.e., they are both cold and heat resistant.

In addition to the additives described, the surfaces of the products and/or their environment, especially the ambient air and/or the surfaces of the equipment or other materials immediately or mediately contacting the products, may be treated with one or more processing aids containing at least two flavoring agents prior to, during or after completion of the process for the manufacturing, processing or packaging of the products, i.e., in addition to using the additives, an external treatment with processing aids may also be performed according to the invention.

The flavoring agents contained in the processing aids are exclusively natural or synthetic (but identical with naturally occurring) flavoring agents which have been recognized as safe according to the FEMA (GRAS—generally recognized as safe). The mentioned list is the FEMA/FDA GRAS Flavour Substances Lists GRAS 3-15 No. 2001-3815 (as of 1997) which includes natural and synthetic flavoring agents approved by the American public health authority, FDA, for use in foods (FDA Regulation 21 CFR 172.515 (Synthetic Flavoring Substances and Adjuvants) and FDA Regulation 21 CFR 182.20 (Natural Flavoring Substances and Adjuvants)). The flavoring agents which meet these FDA standards may be used “quantum satis”, i.e., they may be contained in the food up to the maximum concentration in which they do not cause any adverse effects on the smell or taste of the food to which they are added. The flavoring agents listed according to the FEMA are largely identical with the substances contained in the corresponding European standard COE.

In addition, the flavoring agents classified as “NAT4” according to Article V European Community Directive Flavourings (Jun. 22, 1988) may also be used according to the invention, provided they are considered safe according to the above mentioned FEMA GRAS list. NAT4 substances are substances which are to be declared as “synthetic, but identical with naturally occurring substances” under certain circumstances, e.g., if these substances are used in connection with and as a component of a natural or synthetic (but identical with a naturally occurring) flavoring agent.

A particular advantage of the processing aids is the fact that they may be added to foods without hesitation in a “quantum satis” concentration range due to their components which are listed in the FEMA GRAS list and have been recognized as safe by the US public health authority FDA, which is perhaps the most critical public health authority of all.

Another particular advantage is that the processing aids do not affect the smell and taste of the treated products.

The processing aids according to the invention are employed, for example, in the form of lubricants, emulsifiers and cleaning agents, spraying media, atomizing media, gas-phase active agents, heat transfer media, and cutting media. The processing aids may also be employed as additives to the agents mentioned.

It is essential that the processing aids are not added to or mixed with the food products. Rather, only the surfaces or cuts of the food products are treated with the processing aids. This may be done by directly treating the surfaces or cuts of the food products with the processing aids. However, it is also possible to add the processing aids to the surfaces of equipment, production machines, packaging means, conveying means, packaging materials and the ambient air.

It is surprising that the microbicidal activity of the processing aids can be seen already when low concentrations are used. When applied to food products, only from 0.01 to 5 g/kg, preferably from 0.05 to 2 g/kg, more preferably from 0.05 to 1 g/kg of food is used. When used for the ambient air, for example, only from 0.001 to 10 g/m³ of air is used. For the surfaces of equipment, even as little as from 0.000001 g to 0.1 g/cm² surface area is used.

If these concentrations are adhered to, the quantities detectable in the food products are only around 0.001% by weight. In contrast, according to the prior art, from 0.1 to 3% by weight of preservative will be present in the food products as a rule. It is surprising that, in spite of these extremely low concentrations, an increase in storage life of up to 50% can be achieved according to the invention as compared to conventionally preserved foods.

It is to be pointed out particularly and astonishing that as little as 0.001% by weight of processing aids which are indirectly applied to food products is sufficient to achieve a stabilization or improvement of the keeping quality while the product quality is increased.

This effect is all the more surprising since the duration of the microbicidal action of the flavoring agents used according to the invention is less than 24 hours, preferably less than 12 hours. It is particularly preferred to select such processing aids and concentrations that the duration of microbicidal action is less than 1 hour, preferably less than 15 minutes.

In contrast, conventional preservatives are supposed to be active in the food product as long as possible, i.e., for weeks and months. In spite of the very short duration of action of the processing aids used according to the invention (exposure time), the keeping quality or storage life is significantly increased as compared to food products treated with conventional preservatives or preservation methods according to the prior art. Consequently, according to the invention, when the above described additives and the processing aid are combined, it is possible, surprisingly, to work with significantly lower amounts than that required when the preservatives which have been usual in the prior art are employed.

The processing aid which may be used according to the invention includes flavoring agents selected from the group consisting of alcohols, aldehydes, phenols, acetates, acids, esters, terpenes, acetals and their physiologically acceptable salts, essential oils and plant extracts.

Preferred embodiments of the processing aids according to the invention include one or more flavoring agents selected from one or more of the following groups:

I. Alcohols

Acetoin (acetylmethylcarbinol), ethyl alcohol (ethanol), propyl alcohol (1-propanol), iso-propyl alcohol (2-propanol, isopropanol), propylene glycol, glycerol, benzyl alcohol, n-butyl alcohol (n-propyl carbinol), iso-butyl alcohol (2-methyl-1-propanol), hexyl alcohol (hexanol), L-menthol, octyl alcohol (n-octanol), cinnamyl alcohol (3-phenyl-2-propene-1-ol), α-methylbenzyl alcohol (1-phenylethanol), heptyl alcohol (heptanol), n-amyl alcohol (1-pentanol), iso-amyl alcohol (3-methyl-1-butanol), anisic alcohol (4-methoxybenzyl alcohol, p-anisic alcohol), citronellol, n-decyl alcohol (n-decanol), geraniol, β-γ-hexenol (3-hexenol), lauryl alcohol (dodecanol), linalool, nerolidol, nonadieneol (2,6-nonadiene-1-ol), nonyl alcohol (nonanol-1), rhodinol, terpineol, borneol, clineol (eucalyptol), anisole, cuminyl alcohol (cuminol), 10-undecene-1-ol 1-hexadecanol.

II. Aldehydes

Acetaldehyde, anisic aldehyde, benzaldehyde, iso-butyl aldehyde (methyl-1-propanal), citral, citronellal, n-capraldehyde (n-decanal), ethylvanillin, furfurol, heliotropin (piperonal), heptyl aldehyde (heptanal), hexyl aldehyde (hexanal), 2-hexenal (β-propylacrolein), hydrocinnamic aldehyde (3-phenyl-1-propanal), lauryl aldehyde (dodecanal), nonyl aldehyde (n-nonanal), octyl aldehyde (n-octanal), phenylacetaldehyde (1-oxo-2-phenylethane), propionaldehyde (propanal), vanillin, cinnamic aldehyde (3-phenylpropenal), perillaldehyde, cuminaldehyde.

III. Phenols

Thymol, methyleugenol, acetyleugenol, safrol, eugenol, isoeugenol, anethole, phenol, methylchavicol (estragol; 3-4-methoxyphenyl- 1-propene), carvacrol, α-bisabolol, fornesol, anisole (methoxybenzene), propenylguaethol (5-propenyl-2-ethoxyphenol).

IV. Acetates

Iso-amyl acetate (3-methyl-1-butyl acetate), benzyl acetate, benzylphenyl acetate, n-butyl acetate, cinnamyl acetate (3-phenylpropenyl acetate), citronellyl acetate, ethyl acetate (acetic ester), eugenol acetate (acetyleugenol), geranyl acetate, hexyl acetate (hexanyl ethanoate), hydrocinnamyl acetate (3-phenylpropyl acetate), linalyl acetate, octyl acetate, phenylethyl acetate, terpinyl acetate, triacetin (glyceryl triacetate), potassium acetate, sodium acetate, sodium diacetate, calcium acetate.

V. Acids and/or their physiologically acceptable salts

Acetic acid, aconitic acid, adipic acid, formic acid, malic acid (1-hydroxysuccinic acid), capronic acid, hydrocinnamic acid (3-phenyl-1-propionic acid), pelargonic acid (nonanoic acid), lactic acid (2-hydroxypropionic acid), phenoxyacetic acid (glycolic acid phenyl ether), phenylacetic acid (α-toluenic acid), valeric acid (pentanoic acid), iso-valeric acid (3-methylbutyric acid), cinnamic acid (3-phenylpropenoic acid), citric acid, mandelic acid (hydroxyphenylacetic acid), tartaric acid (2,3-dihydroxybutanedioic acid; 2,3-dihydroxysuccinic acid), fumaric acid, tannic acid.

VI. Esters

Allicin.

VII. Terpenes

Camphor, limonene, β-caryophyllene.

VIII. Acetals

Acetal, acetaldehyde dibutyl acetal, acetaldehyde dipropyl acetal, acetaldehyde phenethyl propyl acetal, cinnamic aldehyde ethylene glycol acetal, decanal dimethyl acetal, heptanal dimethyl acetal, heptanal glyceryl acetal, benzaldehyde propylene glycol acetal.

IX. Polyphenol

X. Essential oils and/or alcoholic or glycolic extracts or extracts obtained by CO₂ high pressure methods from the plants listed below:

a) oils or extracts having a high content of alcohols: melissa, coriander, cardamon, eucalyptus;

b) oils or extracts having a high content of aldehydes: Eucalyptus citriodora, cinnamon, lemon, lemon grass, melissa, citronella, lime, orange;

c) oils or extracts having a high content of phenols: origanum, thyme, rosemary, orange, clove, fennel, camphor, mandarin, anise, cascarilla, estragon and pimento;

d) oils or extracts having a high content of acetates: lavender;

e) oils or extracts having a high content of esters: mustard, onion, garlic;

f) oils or extracts having a high content of terpenes: pepper, bitter orange, caraway, dill, lemon, peppermint, nutmeg apple.

If the processing aid contains only one of the flavoring agents mentioned, isopropanol and ethanol are not employed. Surprisingly, it has been shown that a combination of at least two of the flavoring agents set forth above has a significantly higher effect than one single substance.

Most of the flavoring agents included in the GRAS FEMA list are not water-soluble, i.e., they are hydrophobic. If employed in food products which predominantly contain lipids, they can be directly used without a solvent due to their lipophilic character. However, the proportion of lipophilic foods is relatively low. In order to be able to display their activity in the mostly hydrophilic food or feed products, cosmetics or pharmaceuticals, they are preferably used in connection with a water-soluble solubilizer. In order to meet the requirement of this invention, to provide physiologically safe processing aids, only those solubilizing flavoring agents are used which are approved for use in food, e.g., alcohols.

The processing aids are used neat and/or in water-soluble dilutions with water and/or solvents acceptable for foods (e.g., alcohols) and/or in lipid-soluble dilutions with vegetable (fat) oils.

For example, in the processing aids, there may be used readily water-soluble alcohols, preferably in concentrations of from 0.1 to 99% by weight, based on the processing aid, in connection with other flavoring agents. The processing aids may contain more than 50% of benzyl alcohol. Preferably, they contain less than 50% by weight of ethanol, isopropanol or benzyl alcohol, or of a mixture of these substances. It is particularly preferred for the proportion of the mentioned alcohols to be less than 30% by weight, especially less than 20% by weight. If processing aids are employed which contain benzyl alcohol and at least one other flavoring agent, the proportion of benzyl alcohol may also be more than 50% by weight.

Surprisingly, the processing aids which contain, for example, only 20% by weight of ethanol or isopropanol in connection with flavoring aldehydes and phenols in concentrations in the per mile range have very potent fungicidal and bactericidal activities; even processing aids which contain 1% by weight of the mentioned water-soluble alcohols in connection with less than 3o/oo of flavoring aldehyde and phenol have a 70 to 100% microbicidal activity.

From the above, it can be seen that the processing aids according to the invention have surprising microbicidal effects on the environment of the production or the production process.

Preferred is the use of the processing aids for the production in food and feed products, cosmetics, pharmaceuticals, paints, papers and/or pulps.

In particularly preferred embodiments, the processing aids are used for the improvement and stabilization of the keeping quality of food products selected from the following group:

Breads, pastries, improvers, baking powders, blancmange powders, beverages, dietic food products, essences, delicatessen, fish and fish products, potatoes and products based on potatoes, spices, flours and meals, margarine, fruits and vegetables and products based on fruits and vegetables, pickled preserves, starch products, sweets, soups, pasta food, meat and meat products, milk, dairy and cheese products, poultry and poultry products, oils, fats and products containing oils or fats.

The processing aid displays its activity in the environment of the perishable product, for example, a food or feed product, e.g., on machine parts which contact the product to be worked or processed, or in the air. Due to the direct contact with the surface of the perishable product, they display their activity there as well, i.e., they display their activity on the surface or, when introduced in the product, within the product itself.

Therefore, a particular advantage of the processing aid described is that it is a reliable decontaminant, on one hand, its effectivity against Gram-positive and Gram-negative bacteria, fungi including yeasts and also viruses having been proven, while on the other hand, it is no danger to the consumer of the food since it is absolutely harmless towards him and has no microbicidal technological after-effect in the food, because its microbicidal activity relates to the production environment which is freed from contaminating microorganisms by the measures according to the invention.

The processing aid to be used according to the invention may be a lubricant which at the same time serves for lubrication, decontamination of the lubricated parts and thus indirectly for the keeping quality stabilization of the products contacting these parts.

Further, according to the invention, the processing aid may be an emulsifier, release or cleaning agent. Such agents serve for emulsifying and/or cleaning and thus also for the decontamination of surfaces, objects, machines, devices, equipment, cutting surfaces or devices, conveying devices and the like. In addition, it may be used for decontaminating and cleaning foods, raw materials, cosmetics, pharmaceuticals, paints, papers, pulps, livestock, poultry, fish and waste.

The processing aid to be used according to the invention may also be a spraying medium. Such a spraying medium enables finely distributing the decontaminating agents on all machine parts, conveying devices, cutting devices, working surfaces etc., and it may at the same time have the result that foods packaged immediately after the cutting process and/or packaging-portioning process are stored in a climate having decontaminating and/or keeping quality stabilizing properties due to entrapped spraying agent. In addition, atomizable or sprayable embodiments are very inexpensive due to the necessary amount's being comparably lower.

Similarly, the spraying agent may be blown or sprayed/atomized into and/or onto packages, such as bags, cardboard boxes or the like in order to thereby confer to the product packaged therein a longer storage life.

The spraying agents also serve for atomizing in the surroundings of the production (environment, cooling system, air supply, fresh air) at hygienically weak points (e.g., cooling lines), thereby to reduce the number of germs without causing damage to the people working there.

The processing aids may also be employed for spraying onto food surfaces or cuts in order to eliminate or reduce the perishing-causing agents present on the foods.

Further, these spraying agents may be employed in conveying means, storage and cooling rooms and the like.

In another embodiment, the processing aid is a gas-phase active agent which serves for the active decontamination and/or deodorization in the gas phase within more or less closed systems, such as packages, waste systems, container systems, shipping or storage rooms and the like. Both the packaged product contained, shipped or stored in the container and the air and the respective environment benefit from the activity of the gas-phase agent.

In addition, the processing aid has proven to be a good heat transfer medium. “Heat transfer medium” means cooling, heating and warming media which can be used as decontaminating additives in circulating systems of liquid cooling, heating and warming systems. They are added to aqueous or oily systems for preventing the growth of microorganisms in the liquids in order to prevent contamination, for example, when cooling systems are leaking.

In another embodiment, the processing aid is a cutting medium for cutting blades and/or cutting devices of all kinds and for all perishable products to be cut in order to prevent contamination of the cutting surfaces.

In the food industry, contaminations by Gram-negative or Gram-Positive pathogens, molds, yeasts and other possible perishing-causing agents are often observed on the cutting surfaces of foods, which may reduce the storage life of the cut products, and in part substantially so, and thus cause damage in terms of both health and economy. The contaminations are imported by raw materials, residues of products/raw materials, workers, machine parts or operational processes or by the air.

Therefore, conventionally, either the cut foods or foods to be cut are pasteurized or technically treated to decontamine them and thus confer to them a higher storage life, or else preservatives are added. However, as mentioned above, a thermal treatment is not always possible or allowable and may lead to a reduction of the product quality.

An accessory measure for improving the keeping quality of foods is the cleaning or even disinfection of the environment using chemical disinfectants, which treatment is subject to the biocide regulation. These substances are more or less toxic and must not be transferred into food products. However, chemical disinfection is a discontinuous measure which can be performed, in practice, only at certain production times for machine parts and in the environment, after which rinsing with water for removing any residual substances is required. Accordingly, a direct permanent elimination of perishing-causing agents cannot be ensured.

Therefore, in the prior art, it has been tried to optimize the machine hygiene through better cleanability or through installations for the production or maintenance of pure or low-germ or germ-free air. However, experience has shown that this did not cause an increased keeping quality of cut food products, or it is no longer reasonable economically, or it cannot be safely performed in practice.

An example from the sliced bread industry shows that the cutting or slicing of breads such as whole-dough, whole-meal, white, wheat-and-rye bread or bread for toasting followed by packaging substantially reduces the storage life of the sliced bread as compared to unsliced bread. Depending on the kind of bread, it is between 2 and 5 days. The subsequent thermal treatment (pasteurizing in ovens or microwave ovens at a core temperature of 60 to 90° C.), which is mostly performed today, normally extends the storage life of bread up to from 4 to about 20 days if normal vapor-permeable polyethylene bag packages are used. Other plastic sheets, e.g., of polypropylene, which are substantially more expensive, however, can achieve a longer storage life due to their lower vapor permeability. Packages with polyester plastic materials and an introduced nitrogen-containing atmosphere result in an even longer storage life. However, all these measures either are very expensive, or they can be applied only to expensive special products and markets, and in part they lead to significant quality losses of the sliced bread, e.g., through condensate formation in the bread bag, too soft bread consistencies or premature drying. None of these measures affect the actual causes of contamination by the cutting process, which introduces or distributes the potential causative agents of perishing, present in the environment as well as in the product or on the machine, in the food product via the cutting device, e.g., the cutting blades.

As cutting aids, there are usually employed either mineral compositions, which are no longer allowable in many countries, or vegetable cutting oils which are often inherently contaminated, i.e., bacterially loaded. See, for example, G. Schuster: Investigations on mould contamination of sliced bread, Bäcker & Konditor 27(11), p. 345-347; G. Spicher: Die Quellen der direkten Kontamination des Brotes mit Schimmelpilzen; Das Schneidöl als Faktor der Schimmelkontamination; Getreide, Mehl und Brot 32(4), p.91-94.

Therefore, there is an urgent need for a cutting medium which allows for a decontamination of the machine parts contacting the food product during the cutting process and thereby causes an improved keeping quality of the cut product; this need is satisfied by the cutting medium according to the invention.

The cutting medium can be employed wherever industrial cutting is performed and the cut product can be subject to perishing from bacteria or fungi or to a contamination by viruses. This applies, for example, to pulps and papers, but particularly to food or feed products.

In a preferred embodiment, the processing aid according to the invention is useful for the cutting or slicing of bread, pastries, fish and fish products, potatoes and products based on potatoes, fruits and vegetables and products based on fruits and vegetables, sweets, starch products, pasta food, meat and meat products, cheese products, poultry and poultry products.

If the processing aid is a cutting or slicing medium (e.g., for the slicing of bread), it may be provided with a usual base of vegetable oil/fat/wax with the addition of microbicidal processing aids based on flavoring agents. According to the invention, the cutting medium (e.g., for use in the meat products industry) may preferably consist exclusively of one or more flavoring agents.

Natural emulsifiers, e.g., lecithins, may also be added to the vegetable oils, waxes and fats in a concentration of from 1 to ²5% by weight, in accordance with the prior art. Exemplary emulsifiers are lecithins, citric acid monoglycerides, diacetyltartaric acid, N-acetylphosphatidyl ethanolamine, phosphatidyl inositol, phosphatidyl serine, phosphatide acids, phosphatidyl choline. However, if the cutting medium according to the invention is provided as an aqueous-based emulsion, there are used vegetable oils, vegetable fats and vegetable waxes with unsaturated and saturated C₁₆-C₁₈ fatty acids which also have a viscosity of about 10 mPa·s (20° C.) to about 500 mPa·s (20° C.).

The cutting medium, which is composed, for instance, of the above mentioned fatty acids or oils and emulsifiers, can be employed as a cutting emulsion (milk) after mixing with water in a ratio of from 1:1 to 1:40.

In practice, the cutting medium is applied to at least the machine parts contacting the product to be cut for decontaminating them. According to former experience, the media are suitably employed in dosages of 1-20 g/kg of food, the dosage depending on the cutting device employed and the kind of product to be cut.

The cutting media are mostly applied to the cutting devices, e.g., in bread slicing, they are sprayed onto circular knife slicing machines by means of which bread is subsequently sliced. According to the invention, parts of the cutting devices, e.g., circular knives, band slicers (rotating band saws), electrical or mechanical knives or cutting devices, electrical or mechanical saws or sawing devices, electrical or mechanical chain saws or devices, are wetted so that the cutting medium can display its decontaminating or microbicidal activity on the respective machine part and also on the surface generated by the cutting or slicing.

The beneficial effect of the cutting media to be used according to the invention is manifested by a prolonged storage life of the cut product, e.g., sliced bread. Not least, it is based on the fact that the cutting medium penetrates the surface of the cut product and also decontaminates the bulk of the cut food product, namely through the flavoring agents contained in the cutting oil.

In addition, the flavoring agents herein described have microbicidal activity in the vapor phase since most flavoring agents are easily volatilized. Therefore, they are active in the so-called surroundings of the food product, e.g., within the package of the food product when it has been packaged after the cutting process, e.g., in a plastic sheet package.

This process of decontamination of the cut product after the actual cutting process can be enhanced by a gentle thermal aftertreatment of the cut product.

In the following, the invention will be further described by Examples:

Bacteriological assay methods for additives

quantitative suspension assay I (germ carrier test)

quantitative suspension assay II (suspension test)

quantitative suspension assay III (agar diffusion test)

Microorganisms: aerobic microorganisms (total germ count), Enterobacteriaceae, Enterococci, Lactobacilli, yeasts, molds. These methods are capable of determining the effects of the additives as a function of dosage and duration of action using different microorganisms on different nutrient media.

Quantitative suspension assay I—germ carrier test

Suspension, depending on the germ being tested:

Ringer solution

Tryptone soybean broth

Chromcult Enterococcus broth

wort broth

Germ carrier: 5×5 cm autoclaved cotton cloth or filter

Nutrient agar:

total aerobics <plate count agar

(casein peptone glucose yeast extract agar)

Chromocult

<Enterococcus faecalis

Enterococcus faecium

Streptococcus bovis

QGYE selective nutrient medium (yeast extract—glucose—oxytetracyclin)

Microorganisms—molds

Aspergillus niger

Saccharomyces

Deoxycholate lactose agar

Microorganisms

Lactose-positive—Escherichia coli

Lactose slightly positive—Enterobacter (cloacae)

Lactose slightly positive—Klebsiella (pneumoniae)

Lactose-negative—Salmonella (typhimurium and enteritidis)

Lactose-negative—Shigella (flexneri)

Lactose-negative—Proteus (mirabilis)

Lactose-negative—Pseudomonas

Lactose-negative—Enterococcus (faecalis)

MRS-AGAR (Lactobacillus)

Lactobacillus vulgaris

Baird-Parker agar (with eggyolk-tellurite emulsion)

Microorganisms

Staphylococcus aureus

Staphylococcus epidermidis

Micrococcus (Enterococcus faecium)

Bacillus subtilis

Yeasts: Endomyces tibuliger

Cereus Mossel's selective agar (with eggyolk emulsion)

Microorganisms

Bacillus cereus

Bacillus subtilis

Escherichia coli

Pseudomonas aeruginosa

Proteus mirabilis

Staphylococcus aureus

Deoxycholate lactose agar

Microorganisms

Lactose-positive—Escherichia coli

Lactose slightly positive—Enterobacter (cloacae)

Lactose slightly positive—Klebsiella (pneumoniae)

Lactose-negative—Salmonella (typhimurium and enteritidis)

Lactose-negative—Shigella (flexneri)

Lactose-negative—Proteus (mirabilis)

Lactose-negative—Pseudomonas (Enterococcus faecalis)

TGE agar (casein peptone-qlucose-meat extract agar)

Microorganisms

Staphylococcus aureus

Streptococcus agalactiae

Enterococcus faecalis

Escherichia coll

Salmonella typhimurium

Pseudomonas aeruginosa

Bacillus cereus

Suspension assay—quantitative germ carrier test

Others: special nutrient media and differentiations

for: Clostridia, Listeria and others

Contamination of the germ carriers

The contamination of the germ carriers is effected after placing in a sterile glass dish by pouring the test germ suspension (≧10⁶/ml) over them.

After 1-10 min of storing, the germ carriers are distributed in a glass dish covered with sterile filter paper, and dried in an incubator at 36° C.±1° C.

Test

The contaminated and dried germ carriers are placed in sterile glass dishes and soaked with (gr. %/formulation) each; stored for 1 h and placed for the respectively designated agar/test germ and incubated in the incubator at the prescribed temperature.

After the recommended incubation time, the germ carriers are diluted in a 9-fold dilution (depending on the test germ) of 10¹ to 10⁸, and introduced in the respectively designated agar by a plate casting method.

Calculation

All having from to 200 colonies as determined using the weighed arithmetic mean:

$$\overline{C} = \frac{\sum c}{{n}_{1} \times 1 + {n}_{2} \times 0.1} \times d$$

\overline{C}=number of colony-forming units per ml/g

Σc=sum of the colonies from all Petri dishes used for the calculation

n₁=number of Petri dishes of the lowest dilution stage used for the calculation (n₁=2 for 2 Petri dishes)

n₂=number of Petri dishes of the next higher dilution stage used for the calculation

d=factor of the lowest evaluated dilution stage, the dilution stage related to n₁.

Quantitative suspension assay II—suspension test

a) Inoculate test germ suspension with the desired test germ, e.g., 10⁶/ml, expose for 1-60 min. Place desired formulation to be tested into desiganted germ suspension tubes (different percentages). Await end of exposure times and pour or inoculate into the appropriate agar plates, depending on the germ.

b) Treat the test germ suspension with the desired formulation to be tested (see a) prior to inoculating the test germs (see a). Await end of exposure times and then inoculate with the respective test germs, and inoculate or pour into the appropriate agar plates depending on the test germ.

Quantitative suspension assay III—agar diffusion test

Cast nutrient agar plates which contain, e.g., 10⁴ microorganisms/ml.

A sterile filter paper slip (10 mm) is soaked with the formulation to be tested and placed on the nutrient agar plate.

After the incubation of (time/temperature depending on the germ), the formation of an inhibition halo is read as a positive response.

Formulation Examples
Bacteriological Effectivity

| Lactic acid | Benzyl alcohol | Glycerol | Propylene glycol | Prior art - examples | Formulation |
|---|---|---|---|---|---|
| 10⁷ | 10⁸ | 10⁶ | 10⁹ | 5 min exp. time | total germ count |
| 10⁷ | 10⁷ | 10⁸ | 10⁹ | 15 min exp. time | |
| 10⁷ | 10⁷ | 10⁸ | 10⁹ | 60 min exp. time | |
| | | | | 10⁸/ml | control |
| 10⁶ | 10⁷ | 10⁷ | 10⁸ | 5 min exp. time | Enterobacteria |
| 10⁶ | 10⁷ | 10⁷ | 10⁸ | 15 min exp. time | |
| 10⁶ | 10⁷ | 10⁷ | 10⁸ | 60 min exp. time | |
| | | | | 10⁸/ml | control |
| 10⁷ | 10⁸ | 10⁸ | 10⁸ | 5 min exp. time | Enterococci |
| 10⁷ | 10⁷ | 10⁸ | 10⁸ | 15 min exp. time | |
| 10⁷ | 10⁷ | 10⁸ | 10⁸ | 60 min exp. time | |
| | | | | 10⁸/ml | control |
| 10⁵ | 10⁵ | 10⁵ | 10⁵ | 5 min exp. time | Lactobacilli |
| 10⁴ | 10⁵ | 10⁵ | 10⁵ | 15 min exp. time | |
| 10⁴ | 10⁵ | 10⁵ | 10⁵ | 60 min exp. time | |
| | | | | 10⁵/ml | control |
| 10⁵ | 10⁵ | 10⁵ | 10⁵ | 5 min exp. time | yeasts |
| 10⁵ | 10⁴ | 10⁵ | 10⁵ | 15 min exp. time | |
| 10⁵ | 10⁴ | 10⁵ | 10⁵ | 60 min exp. time | |
| | | | | 10⁵/ml | control |
| 10⁵ | 10⁵ | 10⁵ | 10⁵ | 5 min exp. time | molds |
| 10⁵ | 10⁴ | 10⁵ | 10⁵ | 15 min exp. time | |
| 10⁵ | 10⁴ | 10⁵ | 10⁵ | 60 min exp. time | |
| | | | | 10⁵/ml | control |

| 3 tannin 1 part | 2 tannin 1 part | 1b tannin 1 part | | | |
|---|---|---|---|---|---|
| benzyl alcohol | benzyl alcohol | benzyl alcohol | 1a | | |
| 100 parts | 3 parts | 1 part | tannin | Invention - Examples - 1a 1b | Formulation |
| 10³ | 10² | 10² | 10⁶ | 5 min exp. time | total germ count |
| 10³ | 10² | 10² | 10⁶ | 15 min exp. time | |
| 10³ | 10² | 10³ | 10⁶ | 60 min exp. time | |
| | | | | 10⁸/ml | control |
| 10² | 10¹ | 10² | 10⁶ | 5 min exp. time | Enterobacteria |
| 10² | 10¹ | 10¹ | 10⁶ | 15 min exp. time | |
| 10² | 10¹ | 10¹ | 10⁶ | 60 min exp. time | |
| | | | | 10⁸/ml | control |
| 10³ | 10⁴ | 10³ | 10⁷ | 5 min exp. time | Enterococci |
| 10² | 10³ | 10² | 10⁶ | 15 min exp. time | |
| 10³ | 10² | 10¹ | 10⁶ | 60 min exp. time | |
| | | | | 10⁸/ml | control |
| 10² | 10¹ | 10² | 10⁵ | 5 min exp. time | Lactobacilli |
| 10² | 10¹ | 10¹ | 10⁴ | 15 min exp. time | |
| 10² | 10¹ | 10¹ | 10⁴ | 60 min exp. time | |
| | | | | 10⁵/ml | control |
| 10² | 10¹ | 10¹ | 10⁴ | 5 min exp. time | yeasts |
| 10² | 10¹ | 10¹ | 10⁴ | 15 min exp. time | |
| 10¹ | 10¹ | 10¹ | 10⁴ | 60 min exp. time | |
| | | | | 10⁵/ml | control |
| 10¹ | 10¹ | 10¹ | 10⁴ | 5 min exp. time | molds |
| 10² | 10¹ | 10¹ | 10⁴ | 15 min exp. time | |
| 10¹ | 10¹ | 10¹ | 10⁴ | 60 min exp. time | |
| | | | | 10⁵/ml | control |

| 9 | 8 | 7 | 6 | 5 | 4 | | |
|---|---|---|---|---|---|---|---|
| tannin | tannin | tannin | tannin | tannin | tannin | | |
| 1000 parts | 1000 parts | 100 parts | 3 parts | 1 part | 1 part | | |
| benzyl alcohol | benzyl alcohol | benzyl alcohol | benzyl alcohol | benzyl alcohol | benzyl alcohol | Invention - | Formula- |
| 1 part | 1 part | 1 part | 1 part | 10000 parts | 1000 parts | Example e | tion |
| 10⁵ | 10⁴ | 10⁴ | 10³ | 10³ | 10³ | 5 min exp. time | total |
| 10⁴ | 10⁵ | 10⁴ | 10³ | 10³ | 10³ | 15 min exp. time | germ |
| 10⁴ | 10⁴ | 10⁴ | 10³ | 10³ | 10⁴ | 60 min exp. time | count |
| | | | | | | 10⁸/ml | control |
| 10⁴ | 10⁴ | 10³ | 10³ | 10³ | 10³ | 5 min exp. time | Entero- |
| 10⁴ | 10³ | 10³ | 10² | 10³ | 10² | 15 min exp. time | bacteria |
| 10³ | 10³ | 10³ | 10² | 10³ | 10² | 60 min exp. time | |
| | | | | | | 10⁸/ml | control |
| 10⁴ | 10⁴ | 10⁴ | 10³ | 10³ | 10⁴ | 5 min exp. time | Entero- |
| 10⁴ | 10⁴ | 10⁵ | 10⁴ | 10³ | 10³ | 15 min exp. time | cocci |
| 10⁴ | 10⁴ | 10⁵ | 10⁴ | 10² | 10³ | 60 min exp. time | |
| | | | | | | 10⁸/ml | control |
| 10⁴ | 10⁴ | 10⁴ | 10³ | 10⁴ | 10³ | 5 min exp. time | Lacto- |
| 10⁴ | 10³ | 10⁴ | 10⁴ | 10³ | 10² | 15 min exp. time | bacilli |
| 10⁴ | 10³ | 10⁴ | 10³ | 10² | 10² | 60 min exp. time | |
| | | | | | | 10⁵/ml | control |
| 10³ | 10³ | 10³ | 10⁴ | 10³ | 10² | 5 min exp. time | yeasts |
| 10³ | 10² | 10³ | 10⁴ | 10² | 10¹ | 15 min exp. time | |
| 10³ | 10² | 10³ | 10³ | 10² | 10¹ | 60 min exp. time | |
| | | | | | | 10⁵/ml | control |
| 10³ | 10⁴ | 10³ | 10³ | 10² | 10¹ | 5 min exp. time | molds |
| 10⁴ | 10⁴ | 10³ | 10³ | 10² | 10¹ | 15 min exp. time | |
| 10⁴ | 10⁴ | 10³ | 10³ | 10¹ | 10¹ | 60 min exp. time | |
| | | | | | | 10⁵/ml | control |

| 11 benzyl alcohol 1 part | 10 benzyl alcohol 1 part | | |
|---|---|---|---|
| propylene glycol 10 parts | propylene glycol 1 part | Invention - Examples - 1b | Formulation |
| 10³ | 10³ | 5 min exp. time | total germ count |
| 10⁴ | 10³ | 15 min exp. time | |
| 10⁴ | 10⁴ | 60 min exp. time | |
| | | 10⁸/ml | control |
| 10³ | 10³ | 5 min exp. time | Enterobacteria |
| 10⁴ | 10³ | 15 min exp. time | |
| 10³ | 10² | 60 min exp. time | |
| | | 10⁸/ml | control |
| 10⁴ | 10⁴ | 5 min exp. time | Enterococci |
| 10⁴ | 10³ | 15 min exp. time | |
| 10⁴ | 10³ | 60 min exp. time | |
| | | 10⁸/ml | control |
| 10⁴ | 10³ | 5 min exp. time | Lactobacilli |
| 10³ | 10³ | 15 min exp. time | |
| 10³ | 10³ | 60 min exp. time | |
| | | 10⁵/ml | control |
| 10⁴ | 10³ | 5 min exp. time | yeasts |
| 10³ | 10³ | 15 min exp. time | |
| 10³ | 10³ | 60 min exp. time | |
| | | 10⁵/ml | control |
| 10³ | 10³ | 5 min exp. time | molds |
| 10³ | 10³ | 15 min exp. time | |
| 10³ | 10³ | 60 min exp. time | |
| | | 10⁵/ml | control |

| 14 benzyl alcohol 1 part | 13 benzyl alc. 1 part | 12 benzyl alc. 1 part | Invention - | |
|---|---|---|---|---|
| propylene glycol 10000 parts | propylene glycol 1000 parts | propylene glycol 100 parts | Examples - 1b | Formulation |
| 10⁵ | 10⁵ | 10⁴ | 5 min exp. time | total germ count |
| 10⁵ | 10⁴ | 10⁴ | 15 min exp. time | |
| 10⁵ | 10⁴ | 10⁵ | 60 min exp. time | |
| | | | 10⁸/ml | control |
| 10⁵ | 10⁵ | 10⁴ | 5 min exp. time | Enterobacteria |
| 10⁵ | 10⁴ | 10⁴ | 15 min exp. time | |
| 10⁴ | 10⁴ | 10⁴ | 60 min exp. time | |
| | | | 10⁸/ml | control |
| 10⁵ | 10⁵ | 10⁵ | 5 min exp. time | Enterococci |
| 10⁵ | 10⁵ | 10⁵ | 15 min exp. time | |
| 10⁵ | 10⁵ | 10⁵ | 60 min exp. time | |
| | | | 10⁸/ml | control |
| 10⁴ | 10⁵ | 10⁴ | 5 min exp. time | Lactobacilli |
| 10⁴ | 10⁴ | 10⁴ | 15 min exp. time | |
| 10⁴ | 10⁴ | 10³ | 60 min exp. time | |
| | | | 10⁵/ml | control |
| 10⁴ | 10⁴ | 10⁴ | 5 min exp. time | yeasts |
| 10⁴ | 10⁴ | 10⁴ | 15 min exp. time | |
| 10⁴ | 10⁴ | 10⁴ | 60 min exp. time | |
| | | | 10⁵/ml | control |
| 10⁴ | 10⁴ | 10⁴ | 5 min exp. time | molds |
| 10⁴ | 10⁴ | 10⁴ | 15 min exp. time | |
| 10⁴ | 10³ | 10³ | 60 min exp. time | |
| | | | 10⁵/ml | control |

| 16 benzyl alcohol 100 parts | 15 benzyl alcohol 10 parts | | |
|---|---|---|---|
| glycerol 1 part | glycerol 1 part | Invention - Examples - 1b 1c | Formulation |
| 10⁵ | 10⁴ | 5 min exp. time | total germ count |
| 10⁵ | 10⁴ | 15 min exp. time | |
| 10⁵ | 10⁴ | 60 min exp. time | |
| | | 10⁸/ml | control |
| 10⁵ | 10⁴ | 5 min exp. time | Enterobacteria |
| 10⁴ | 10⁴ | 15 min exp. time | |
| 10⁴ | 10⁴ | 60 min exp. time | |
| | | 10⁸/ml | control |
| 10⁵ | 10⁴ | 5 min exp. time | Enterococci |
| 10⁵ | 10⁴ | 15 min exp. time | |
| 10⁴ | 10⁴ | 60 min exp. time | |
| | | 10⁸/ml | control |
| 10⁴ | 10⁴ | 5 min exp. time | Lactobacilli |
| 10⁴ | 10⁴ | 15 min exp. time | |
| 10⁴ | 10³ | 60 min exp. time | |
| | | 10⁵/ml | control |
| 10⁴ | 10⁴ | 5 min exp. time | yeasts |
| 10⁴ | 10⁴ | 15 min exp. time | |
| 10⁴ | 10³ | 60 min exp. time | |
| | | 10⁵/ml | control |
| 10⁴ | 10⁴ | 5 min exp. time | molds |
| 10⁴ | 10³ | 15 min exp. time | |
| 10⁴ | 10³ | 60 min exp. time | |
| | | 10⁵/ml | control |

| 17 lactic acid 1 part | 18 anisole 1 part | | |
|---|---|---|---|
| tannin 1 part | tannin 1 part | | |
| benzyl alcohol 2 parts | benzyl alcohol 88 parts | Invention - Examples | Formulation |
| 10² | 10³ | 5 min exp. time | total germ count |
| 10¹ | 10³ | 15 min exp. time | |
| 10¹ | 10³ | 60 min exp. time | |
| | | 10⁸/ml | control |
| 10² | 10³ | 5 min exp. time | Enterobacteria |
| 10¹ | 10³ | 15 min exp. time | |
| 10¹ | 10² | 60 min exp. time | |
| | | 10⁸/ml | control |
| 10³ | 10³ | 5 min exp. time | Enterococci |
| 10³ | 10³ | 15 min exp. time | |
| 10² | 10⁴ | 60 min exp. time | |
| | | 10⁸/ml | control |
| 10¹ | 10² | 5 min exp. time | Lactobacilli |
| 10¹ | 10² | 15 min exp. time | |
| 10¹ | 10² | 60 min exp. time | |
| | | 10⁵/ml | control |
| 10¹ | 10³ | 5 min exp. time | yeasts |
| 10¹ | 10² | 15 min exp. time | |
| 10¹ | 10² | 60 min exp. time | |
| | | 10⁵/ml | control |
| 10¹ | 10² | 5 min exp. time | molds |
| 10¹ | 10¹ | 15 min exp. time | |
| 10¹ | 10¹ | 60 min exp. time | |
| | | 10⁵/ml | control |

| 20 tannin 3 parts | 19 tannin 3 parts | | |
|---|---|---|---|
| benzyl alcohol 1 part | benzyl alcohol 1 part | | |
| glycerol 96 parts | Water 96 parts | Invention - Examples - 1e | Formulation |
| 10³ | 10³ | 5 min exp. time | total germ count |
| 10³ | 10³ | 15 min exp. time | |
| 10² | 10³ | 60 min exp. time | |
| | | 10⁸/ml | control |
| 10² | 10³ | 5 min exp. time | Enterobacteria |
| 10² | 10² | 15 min exp. time | |
| 10² | 10² | 60 min exp. time | |
| | | 10⁸/ml | control |
| 10³ | 10³ | 5 min exp. time | Enterococci |
| 10³ | 10³ | 15 min exp. time | |
| 10² | 10³ | 60 min exp. time | |
| | | 10⁸/ml | control |
| 10³ | 10³ | 5 min exp. time | Lactobacilli |
| 10² | 10³ | 15 min exp. time | |
| 10² | 10³ | 60 min exp. time | |
| | | 10⁵/ml | control |
| 10⁴ | 10⁴ | 5 min exp. time | yeasts |
| 10³ | 10⁴ | 15 min exp. time | |
| 10³ | 10³ | 60 min exp. time | |
| | | 10⁵/ml | control |
| 10³ | 10³ | 5 min exp. time | molds |
| 10³ | 10³ | 15 min exp. time | |
| 10² | 10² | 60 min exp. time | |
| | | 10⁵/ml | control |

1. An additive for the improvement and/or stabilization of the keeping quality of microbially perishable products, containing
(i) a mixture of
a) polyphenol and
b) benzyl alcohol and optionally other GRAS (generally recognized as safe) flavor alcohols; or
(ii) a mixture of
c) benzyl alcohol and
d) at least one other GRAS flavor alcohol;
the mixing ratio of components a) to b) or c) to d) being from 1:1 to 1:10,000 or from 10,000:1 to 1:1, wherein said GRAS flavor alcohols are selected from acetoin, ethanol, 1-propanol, 2-propanol, propylene glycol, glycerol, n-butyl alcohol, 2-methyl-1-propanol, hexanol, L-menthol, octyl alcohol, cinnamyl alcohol, 1-phenylethanol, heptanol, 1-pentanol, 3-methyl-1-butanol, 4-methoxybenzyl alcohol, citronellol, n-decanol, geraniol, 3-hexenol, dodecanol, linalool, nerolidol, nonadieneol, nonyl alcohol, rhodinol, terpineol, borneol, clineol, anisole, cuminyl alcohol, 10-undecene-1-ol and 1-hexadecanol.

2. The additive according to claim 1, wherein mixtures (i) and (ii) contain at least one other monohydric or polyhydric alcohol containing from 2 to 10 carbon atoms, preferably from 2 to 7 carbon atoms.

3. The additive according to any of claims 1 or 2, wherein said additive contains at least one organic acid and/or at least one of its physiologically acceptable salts containing from 1 to 15 carbon atoms, preferably from 2 to 10 carbon atoms.

4. The additive according to any of claims 1 to 3, wherein said additive contains phenols, acetates, esters, terpenes, acetals and/or essential oils as components e) to k).

5. The additive according to any of claims 1 to 4, wherein said additive contains a solubilizer, especially glycerol, propylene glycol, water, edible oils or fats, as component 1).

6. The additive according to any of claims 1 to 5, wherein the ratio of components a) to b) or c) to d) is from 1:1 to 1:1000 or from 1000:1 to 1:1, preferably from 1:1 to 1:100 or from 100:1 to 1:1.

7. The additive according to claim 2, wherein the mixing ratio of components a), b), c) and/or d) to said other alcohols is respectively from 1:1 to 1:10,000 or from 10,000:1 to 1:1, preferably from 1:1 to 1:1000 or from 1000:1 to 1: 1.

8. The additive according to any of claims 1 to 7, wherein the mixing ratio of components a) or c) to components e), f), g), h), i), j), k), l) is respectively from 1:1 to 1:10,000 or from 10,000:1 to 1:1, preferably from 1:1 to 1:1000 or from 1000:1 to 1:1.

9. A method for the improvement and/or stabilization of the keeping quality of microbially perishable products, wherein a mixture as defined in any of claims 1 to 8 is added to said microbially perishable product.

10. The method according to claim 9, wherein said additive is added to said microbially perishable product in amounts of from 1 ppm to 10% by weight.

11. The method according to any of claims 9 or 10, wherein said additive is added to said microbially perishable product in amounts of from 0.001% by weight to 0.5% by weight.

12. The method according to any of claims 8 to 11, wherein said additive is added to said microbially perishable product in amounts of from 0.002% by weight to 0.25% by weight.

13. The method according to any of claims 8 to 12, wherein the surfaces of the products and/or their environment, especially the ambient air and/or the surfaces of the equipment or other materials immediately or mediately contacting the products, are treated with one or more processing aids preferably containing at least two microbicidally active flavoring agents prior to, after or during the process for the manufacturing, processing or packaging of the products.

14. The method according to claim 13, wherein said processing aid is used in amounts of from 0.01 to 5 g/kg, preferably from 0.05 to 2 g/kg, of food, or when used for the ambient air, in amounts of from 0.001 to 10 g/m³ of air, or for the surfaces of equipment, in amounts of from 0.000001 g to 0.1 g/cm² surface area.

15. Use of the additive according to any of claims 1 to 8 for the improvement and/or stabilization of the keeping quality of microbially perishable products, especially food products and cosmetics.
